(19) United States
(12) Patent Application Publication
(10) Pub. No.: US 2014/0377542 A1
(43) Pub. Date: Dec. 25, 2014

(54) PLATE WITH TITANIUM DIOXIDE ON ITS SURFACE, MANUFACTURING PROCESS AND USES THEREOF

(71) Applicant: China Medical University, Taichung City (TW)

(72) Inventors: Chao-Jung Chen, Taichung City (TW); Yu-Ching Liu, Taichung City (TW)

(21) Appl. No.: 14/024,902

(22) Filed: Sep. 12, 2013

(30) Foreign Application Priority Data
Jun. 20, 2013 (TW) 102121894

(51) Int. Cl. C07K 14/77; B05D 3/00; C07K 14/47

(52) U.S. Cl. CPC C07K 14/77; C07K 14/4732; B05D 3/007
USPC 428/328; 530/324; 530/326; 530/325; 427/384; 428/447; 428/429; 428/412

(57) ABSTRACT

A plate with titanium dioxide on its surface, as well as its manufacturing process and uses thereof are provided. The plate comprises (A) a substrate; (B) a polydimethylsiloxane (PDMS) layer on at least one surface of the substrate; and (C) one or more aggregations of titanium dioxide particles on the polydimethylsiloxane layer. The plate is useful in the purification of phosphopeptides.

### CLAIM FOR PRIORITY

This application claims the benefit of Taiwan Patent Application No. 102121894, filed on Jun. 20, 2013, in the Taiwan Intellectual Property Office, the disclosure of which is incorporated herein in its entirety by reference.

### CROSS-REFERENCES TO RELATED APPLICATIONS

Not applicable.

### BACKGROUND OF THE INVENTION

1. Field of the Invention

The present invention relates to a plate with titanium dioxide (TiO₂) on its surface, as well as the manufacturing process and uses thereof. The present invention also relates to a method for purifying phosphopeptides, comprising the use of the plate with TiO₂ on its surface.

2. Descriptions of the Related Art

Protein phosphorylation is a common protein post-translational modification, which is a reversible protein modification process. It has been known that protein phosphorylation is relevant to the regulations of many biochemical reactions in an organism, such as the regulation of the growth, metabolism and apoptosis of cells as well as the delivery of signals in cells. The over-phosphorylation of a specific protein in an organism may cause diseases. For example, research has shown that the over-phosphorylation of Tau, a microtubule-associated protein in the human brain, may result in the abnormal protein accumulation in cells which causes neurofibrillary tangles and leads to Alzheimer's disease. Thus, the study of phosphorylated proteins is an important issue in proteomics.

Previous studies on phosphorylated proteins primarily use mass spectrometry (MS) for analyzing the characteristics of a phosphorylated protein and the binding site(s) of the phosphate group(s). However, when identifying the MS signals of the phosphorylated proteins in a protein sample, the signals of extremely low-level phosphopeptides are usually suppressed by those of abundant non-phosphopeptides. Furthermore, due to the increased acidity of phosphopeptides as compared to that of non-phosphopeptides, phosphopeptides are less efficient in the formation of positive ions, and thus, there is much difficulty in identifying phosphopeptides by MS. Therefore, to improve the sensitivity for detecting phosphopeptides by MS, the purification of phosphopeptides from a protein mixture is crucial prior to MS analysis.

One traditional approach for purifying phosphopeptides is immobilized metal ion affinity chromatography (IMAC), which uses metal ions such as Fe³⁺ or Ga³⁺ to chelate the phosphate groups of phosphopeptides. However, because the IMAC approach is based on ionic interactions, its selectivity is usually limited by the non-specific binding of acidic non-phosphopeptides. Another known approach for purifying phosphopeptides is metal oxide affinity chromatography (MOAC), which uses acidic buffers during purification to prevent non-specific binding between non-phosphorylated proteins and ligands.

It has been known that TiO₂ can specifically bind to phosphopeptides. It has been developed to use TiO₂ in an MOAC approach for the manufacture of a pipette tip for purifying phosphopeptides. However, when a traditional pipette tip, such as the TiO₂ Tip, is used to purify phosphopeptides, TiO₂ beads need to be loaded on the pipette tip first, followed by the loading, washing, and elution steps of peptide samples, which are conducted by centrifugation. After the purification procedure, the eluted samples are loaded onto an MS sample plate to perform MS analysis. Therefore, using pipette tips, such as TiO₂ Tips, to purify the phosphopeptides cannot provide a high throughput sample analysis and has the disadvantages of time-consuming, laborious, sample-consuming during multiple processing steps, and experimental reproducibility may be affected by operating variables, such as the rate of sample elution.

The inventors of the present invention found that TiO₂ can be immobilized on one surface by a simple approach, thereby, providing a plate with TiO₂ on its surface. Such plate, when being used in the purification of phosphopeptides, has advantages including easy operation, high throughput (i.e., samples can be purified in a single batch) and high experimental reproducibility, and its substrate can be easily regenerated.

### SUMMARY OF THE INVENTION

An objective of the present invention is to provide a plate with TiO₂ on its surface, comprising the following:

(A) a substrate;

(B) a polydimethylsiloxane (PDMS) layer on at least one surface of the substrate; and

(C) one or more aggregations of TiO₂ particles on the PDMS layer.

Another objective of the present invention is to provide a method of manufacturing the above plate with TiO₂ on its surface, comprising the following steps:
(a) providing a substrate;
(b) forming a PDMS layer on at least one surface of the substrate;
(c) applying an aqueous suspension of TiO₂ particles onto at least a portion of the PDMS layer; and
(d) drying the applied TiO₂ suspension thereby forming one or more aggregations of TiO₂ particles on the PDMS layer.

Yet a further objective of the present invention is to provide a method for purifying phosphopeptides, comprising the following steps:
(i) contacting a phosphopeptide-containing solution with the aforesaid aggregations of TiO₂ particles on the plate with TiO₂ on its surface for a time period ranging from 1 minute to 60 minutes;
(ii) rinsing the aggregations of TiO₂ particles with a wash solution; and
(iii) contacting the aggregations of TiO₂ particles with an elution solution for a time period ranging from 1 minute to 30 minutes to elute phosphopeptide(s).

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The invention is described in detail below with reference to the appended drawings. In the Figures:

FIG. 1a is a photomicrograph showing the aggregations of TiO₂ particles on an embodiment of the plate with TiO₂ particles on its surface of the present invention;

FIG. 1b is an image scanned by an optical scanner showing an embodiment of the plate with TiO₂ on its surface of the present invention;

FIG. 2a is a MALDI-TOF mass spectrum of unpurified ovalbumin;

FIG. 2b is a MALDI-TOF mass spectrum of ovalbumin purified by an embodiment of the plate with TiO₂ particles on its surface of the present invention, wherein the particle size of the TiO₂ particles is about 5 μm;

FIG. 2c is a MALDI-TOF mass spectrum of ovalbumin purified by an embodiment of the plate with TiO₂ particles on its surface of the present invention, wherein the particle size of the TiO₂ particles is smaller than 150 nm;

FIG. 3a is a MALDI-TOF mass spectrum of an unpurified complicated peptides mixture;

FIG. 3b is a MALDI-TOF mass spectrum of a complicated peptides mixture purified by an embodiment of the plate with TiO₂ on its surface of the present invention;

FIG. 4a is a MALDI-TOF mass spectrum of an unpurified phosphopeptides mixture;

FIG. 4b is a MALDI-TOF mass spectrum of a phosphopeptides mixture purified by an embodiment of the plate with TiO₂ on its surface of the present invention;

FIG. 4c is a bar diagram comparing the peak ratios of 1368.68/1046.54 m/z and 1448.64/1046.54 m/z of FIG. 4a and FIG. 4b;

FIG. 5a is a MALDI-TOF mass spectrum of 2 fmole β-casein purified by an embodiment of the plate with TiO₂ on its surface of the present invention;

FIG. 5b is a MALDI-TOF mass spectrum of 20 fmole β-casein purified by a TiO₂ Tip; and

FIG. 6 is a diagram showing the signal intensity ratios of the peak signals (2061.8 m/z to 2465.2 m/z) of β-casein (0.5 μg to 100 μg) purified by an embodiment of the plate with TiO₂ on its surface of the present invention.

### DETAILED DESCRIPTION OF THE INVENTION

The following will describe some embodiments of the present invention in detail. However, without departing from the spirit of the present invention, the present invention may be embodied in various embodiments and should not be limited to the embodiments described in the specification. In addition, unless otherwise state herein, the expressions “a,” “the,” or the like recited in the specification of the present invention (especially in the claims) should include both the singular and the plural forms. Terminology used herein is given its ordinary meaning consistent with the exemplary definitions set forth immediately below.

Protein phosphorylation refers to the attachment of a phosphate (PO₄) group to a protein, which usually occurs on serine, threonine, and/or tyrosine residues. It has been known that because there is an excellent binding affinity between TiO₂ and phosphate groups, TiO₂ can be used for purifying phosphorylated proteins. However, as described above, there are still many disadvantages of the traditional methods used for immobilizing TiO₂ to conduct the purification of phosphorylated proteins. Therefore, there is still a need for a method to immobilize TiO₂ easily and effectively.

The inventors of the present invention coincidentally found that TiO₂ particles can be effectively immobilized on a polydimethylsiloxane (PDMS) layer in an aggregated form through an aqueous suspension of TiO₂ particles, thereby providing a plate with TiO₂ on its surface. The plate is useful in the purification of phosphopeptides. Specifically, the plate with TiO₂ on its surface according to the present invention comprises (A) a substrate; (B) a PDMS layer on at least one surface of the substrate; and (C) one or more aggregations of TiO₂ particles on the PDMS layer.

In the plate with TiO₂ on its surface in accordance with the present invention, the substrate is a component used for supporting the PDMS layer. The shape and material of the substrate can be selected according to practical applications without any particular limitation. In general, PDMS is a material with a good adhesion to various materials. Therefore, the substrate used in the plate of the present invention may be selected from the group consisting of a metal substrate, a glass substrate, a polymer substrate such as a polymethacrylate substrate, a polycarbonate substrate, and a polyethylene terephthalate substrate, and a wood substrate. In addition, the substrate can be flat or non-flat (such as a substrate with groove(s) on its surface) depending on the practical needs. In one embodiment of the present invention, a discoidal stainless steel substrate is used.

PDMS is a polymeric organosilicon compound that is non-flammable, inert, and non-toxic. PDMS is a viscous liquid before curing and has a structure as the following formula (I):

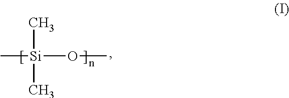

The viscosity of PDMS increases with increments of n values. Part of the methyl group and end group in the structure of PDMS may be substituted by other functional group(s) such as a hydrogen group, a vinyl group, or a phenyl group.

As cured, PDMS is a hydrophobic layer with a good adhesion to various substrates. In addition, cured PDMS is flexible, thermostable, and optically transparent. Cured PDMS can be used in the medical field for artificial organs, guide tubes, contact lenses, and drug delivery systems. In addition, cured PDMS can be used in industry for microfluidic devices and microreactors, or be used as a sealant or an insulator, etc.

In the plate according to the present invention, the PDMS layer can be formed on a surface of a substrate by any method suitable for providing PDMS. For example, the PDMS layer can be formed by mixing a hydrogen-functional PDMS and a vinyl-functional PDMS, and then optionally adding a Pt catalyst, thereby, forming a cured PDMS layer on the substrate. The aforesaid curing reaction is accomplished by an addition reaction between the silane group on the hydrogen-functional PDMS and the vinyl group on vinyl-functional PDMS. Examples of the hydrogen-functional PDMS include, but are not limited to poly(methylhydrosiloxane), trimethylsilyl terminated poly(methylhydrosiloxane), trimethylsilyl terminated poly(dimethylsiloxane-co-methylhydrosiloxane), hydroxy terminated poly(dimethylsiloxane), and hydride terminated poly(dimethylsiloxane). Examples of the vinyl-functional PDMS include, but are not limited to vinyl terminated poly(dimethylsiloxane) and divinyl terminated poly(dimethylsiloxane-co-diphenylsiloxane). Examples of the Pt catalyst include, but are not limited to hexachloroplatinic acid, platinum dioxide, potassium chloroplatinate, platinum dichloride, and platinum tetrachloride.

In addition, a commercial kit for manufacturing a curable PDMS also can be used to provide the PDMS layer of the present invention. In general, a commercial kit of curable PDMS comprises a PDMS elastomer base and a PDMS elastomer curing agent. When using such a commercial kit, the base and the curing agent are usually mixed in a specific ratio to obtain a mixture, and then, the obtained mixture is optionally heated to cure PDMS. In one embodiment of the present invention, commercial Sylgard® 184 organosilicon elastomer kit (Dow Corning Inc., USA), which comprises a PDMS elastomer base (reagent A) and a PDMS elastomer curing agent (reagent B) is used to provide the PDMS layer. The operating steps of using the Sylgard® 184 organosilicon elastomer kit to provide the PDMS layer comprises the following: (1) mixing reagent A and reagent B in a volume ratio ranging from 3:1 to 15:1, and preferably in a volume ratio of about 10:1; (2) applying the obtained mixture solution onto at least one surface of a substrate; and (3) drying the substrate at a temperature ranging from 40° C. to 200° C. for 5 minutes to 120 minutes to form a PDMS layer on the surface of the substrate.

The above Sylgard® 184 organosilicon elastomer kit used in the step (2) for providing the PDMS layer can be applied by any methods known by persons with ordinary knowledge in the art. Suitable applying methods include, but are not limited to a screen-printing method, a coating method or a spotting method. A suitable coating method can be such as a knife coating, a roller coating, a micro gravure coating, a flow coating, a dip coating, a spray coating, a curtain coating, or a combination thereof. For example, a suitable amount of a mixture of the reagent A and reagent B can be applied onto a substrate, and then be optionally smoothed by a glass slide or a roller.

In a plate with TiO₂ on its surface according to the present invention, the thickness of the PDMS layer can be adjusted depending on the practical application without any particular limitations. For instance, one may adjust the volume of the PDMS solution to be coated on a substrate and/or the coating frequency so as to control the thickness of a dried PDMS layer provided thereby.

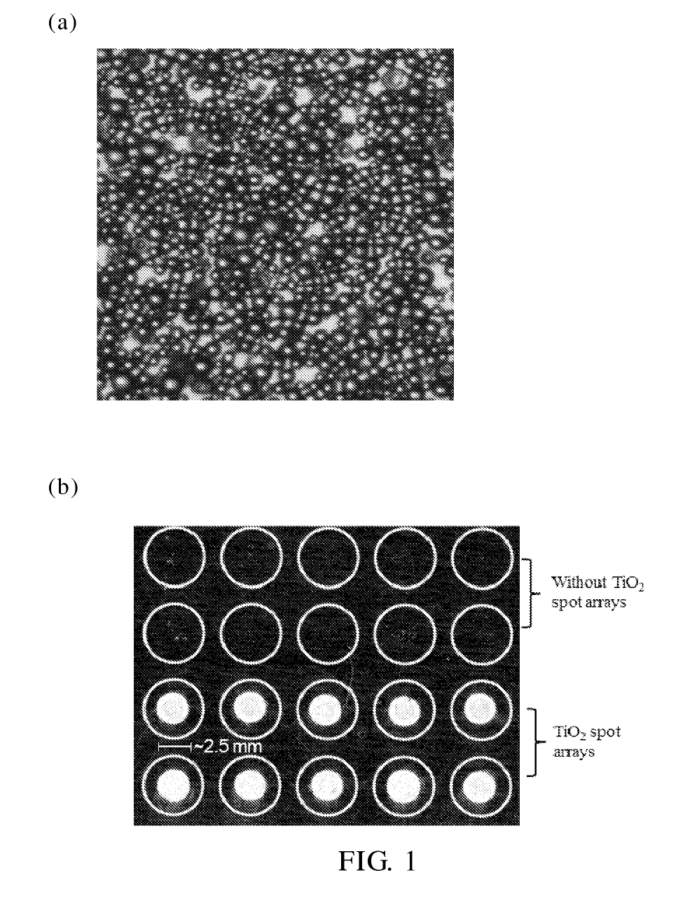

In the plate with TiO₂ on its surface according to the present invention, there are one or more TiO₂ aggregations on the PDMS layer, and each aggregation substantially consists of TiO₂ particles. FIG. 1a shows a photomicrograph of the aggregations of TiO₂ particles on an embodiment of the plate with TiO₂ particles on its surface of the present invention, wherein the particle size of the TiO₂ particles is about 5 μm. FIG. 1b shows an image scanned by an optical scanner of another embodiment of the plate with TiO₂ particles on its surface of the present invention, wherein the TiO₂ particles are distributed as a plurality of aggregations on the surface of the PDMS layer, thereby, rendering the obtained plate with TiO₂ on its surface to conduct the purification of multiple phosphopeptides samples simultaneously.

In the plate with TiO₂ on its surface according to the present invention, the TiO₂ particles in the TiO₂ aggregations have no particular limitations on their particle sizes. However, when the TiO₂ particles are of a nano-scaled particle size such as being smaller than 150 nm, non-specific binding is likely to occur between the provided aggregations of TiO₂ particles and non-phosphopeptides, and this leads to a reduction in specificity for purifying phosphorylated proteins. Therefore, in the plate with TiO₂ on its surface according to the present invention, the TiO₂ particles in the TiO₂ aggregations generally have a particle size of at least 150 nm, preferably from 0.5 μm to 10 μm, and more preferably from 1 μm to 5 μm.

The present invention also provides a method of manufacturing the above plate with TiO₂ on its surface, comprising the following steps: (a) providing a substrate; (b) forming a PDMS layer on at least one surface of the substrate; (c) applying an aqueous suspension of TiO₂ particles onto at least a portion of the PDMS layer; and (d) drying the applied TiO₂ suspension thereby forming one or more aggregations of TiO₂ particles on the PDMS layer. In the method of the present invention, the types and properties of the substrates involved in step (a), the method of manufacturing PDMS layer involved in step (b), and the particle size of the TiO₂ particles in step (c) are as those described herein above for the plate with TiO₂ on its surface according to the present invention.

In step (c) of the method according to the present invention, an aqueous suspension of TiO₂ particles is used, which is crucial to the present invention. The inventors of the present invention unexpectedly found that when an aqueous suspension of TiO₂ particles is applied onto a hydrophobic PDMS layer and dried, the TiO₂ particles can be immobilized on the surface of the PDMS layer. However, if a non-aqueous suspension is used, the desired efficacy of immobilizing TiO₂ cannot be achieved. Any method known by persons with ordinary knowledge in the art can be used to apply the aqueous suspension onto at least a portion of the PDMS layer. For instance, in an embodiment of the present invention, an aqueous suspension of TiO₂ particles was sucked up at an amount of about 2 μl to 5 μl repeatedly by a pipette tip and then dropped onto a PDMS layer to form one or more individual droplets, thereby, forming one or more aggregations consisting of TiO₂ particles on the PDMS layer after drying.

In the procedure for applying an aqueous suspension of TiO₂ particles, the volume of each droplet of the aqueous suspension of TiO₂ particles can be adjusted according to practical application. In general, an aggregation of TiO₂ particles with a larger volume can be obtained after drying by applying the droplets of the TiO₂ aqueous suspension in a larger volume or with an increased concentration of TiO₂ particles in the aqueous suspension. Such larger TiO₂ aggregation, as being used in the purification of phosphopeptides, can enable an increased sample loading capacity.

In addition, in the method according to the present invention, an organic solvent with a polarity lower than that of water can optionally be added to the aqueous suspension of TiO₂ particles to reduce the polarity of the aqueous suspension, reduce the contact angle between the droplets of the aqueous suspension applied on PDMS layer and the PDMS layer, and increase the contact area between the formed aggregations of TiO₂ particles and the PDMS layer. The organic solvent with a polarity lower than that of water can be, but is not limited to, acetonitrile, methanol, ethanol, n-propanol, isopropanol, n-butanol, isobutanol, acrylonitrile, acetone, ethyl acetate, tetrahydrofuran, dichloromethane, trichloromethane, benzene, methyl benzene, n-hexane, n-pentane, or combinations thereof. In some embodiments of the present invention, acetonitrile is added to the aqueous suspension of TiO₂ particles to provide aggregations of TiO₂ particles having a larger contact area with the PDMS layer.

In step (d) of the method according to the present invention, the aqueous suspension of TiO₂ particles or its droplets applied on the PDMS layer can be dried by any suitable approach, such as air drying, baking, or natural drying at room temperature. A high temperature heating procedure is not necessarily required. The aqueous suspension of TiO₂ particles is preferred to be dried at a temperature ranging from 10° C. to 100° C. More preferably, the aqueous suspension of TiO₂ particles is dried at a temperature ranging from 20° C. to 80° C. According to some embodiments of the present invention, the aqueous suspension of TiO₂ particles is dried in an oven at 80° C. or by natural drying at room temperature. Because the method according to the present invention can immobilize TiO₂ onto the PDMS layer by using a low temperature drying step and no high temperature sintering is involved, it is more economical.

As described above, because TiO₂ can bind to phosphate groups on phosphorylated proteins, the plate with TiO₂ on its surface according to the present invention is useful in the purification of phosphopeptides. Therefore, the present invention also provides a method for purifying phosphopeptides by using a plate with TiO₂ on its surface of the present invention, comprising the following steps: (i) contacting a phosphopeptide-containing solution with the aggregations of TiO₂ particles on the plate with TiO₂ on its surface according to the present invention for a time period ranging from 1 minute to 60 minutes; (ii) rinsing the aggregations of TiO₂ particles with a wash solution; and (iii) contacting the aggregations of TiO₂ particles with an elution solution for a time period ranging from 1 minute to 30 minutes to elute the phosphopeptide(s).

Prior to conducting the aforesaid method for purifying phosphopeptides, the surface with aggregation(s) of TiO₂ on it of the plate according to the present invention can optionally be washed with a suitable wash solution such as 0.1% formic acid (FA) and dried to remove contaminants on the surface.

In the above step (i), the phosphopeptide-containing solution may be a hydrolysed protein sample. For example, a protein sample to be purified can be treated by a protease to provide a hydrolysed phosphopeptide-containing solution. A suitable protease includes, but is not limited to trypsin, chymotrypsin, Glu-C protease, Lys-C protease, and Asp-N protease, etc.

After finishing the aforesaid protease treatment, it is preferable that the hydrolysed phosphopeptides are dried and dissolved into a buffer solution first to provide a phosphopetide-containing solution. Then, the phosphopeptide-containing solution comes into contact with the aggregations of TiO₂ particles on the plate with TiO₂ on its surface of the present invention. The contact time preferably ranges from 1 minute to 60 minutes, more preferably from 2 minutes to 20 minutes, to render the phosphopeptides to bind to TiO₂. Preferably, the buffer solution comprises a component selected from the group consisting of acetonitrile (ACN), trifluoroacetic acid (TFA), 2,5-dihydroxybenzoic acid (DHB), and combinations thereof. It has been known that DHB can reduce the electrostatic interaction between acidic amino acid residues (such as asparatic acid and glutamic acid) and TiO₂, and thus, can enhance the binding affinity between phosphate groups on phosphopeptides and TiO₂ particles and reduce non-specific binding reaction.

After finishing the step of contacting the phosphopeptide-containing solution with the aggregations of TiO₂ particles, the aggregations of TiO₂ particles are rinsed with a wash solution to remove non-phosphopeptide(s) which do(es) not bind to TiO₂, i.e. the step (ii). Preferably, the wash solution comprises an organic phase and an acid, wherein the examples of the organic phase include acetonitrile, methanol, ethanol, n-propanol, isopropanol, n-butanol, isobutanol, acrylonitrile, acetone, ethyl acetate, tetrahydrofuran, dichloromethane, trichloromethane, benzene, methyl benzene, n-hexane, n-pentane, and combinations thereof, and the examples of the acid include a substituted or unsubstituted formic acid, a substituted or unsubstituted acetic acid, and combinations thereof. According to one embodiment of the present invention, an aqueous solution comprises 60 wt % to 90 wt % acetonitrile and 1 wt % to 5 wt % TFA is used as the wash solution.

Then, in the above step (iii), an elution solution comes into contact with the aggregation of TiO₂ particles preferably for a time period ranging from 1 minute to 30 minutes, and more preferably for a time period ranging from 2 minutes to 20 minutes, to elute the phosphopeptides. Preferably, the elution solution is selected from the group consisting of an ammonia solution (NH₄OH), an aqueous solution of an ammonium salt (such as ammonium acetate, ammonium carbonate), an aqueous solution of formic acid, and combinations thereof. In one embodiment of the present invention, an ammonia solution is used as the elution solution.

After finishing the eluting procedures of the above step (iii), purified phosphopeptides can be obtained. Herein, depending on the type of the substrate and the purpose of follow-up application, the plate with TiO₂ on its surface according to the present invention (while having the eluted phosphopeptide-containing elution solution on its surface) can be directly analyzed; or the eluted phosphopeptide-containing elution solution is collected first and then the collected elution solution is analyzed. The analysis can be any suitable analysis method including mass spectrometry analysis, liquid chromatography, or a combination of liquid chromatography and mass spectrometry analysis such as LC-MS, HPLC-MS/MS, and nanoLC-MS/MS.

According to an embodiment of the present invention, a MALDI-TOF plate, i.e., a stainless steel substrate is used for the preparation of the plate with TiO₂ on its surface. In this embodiment, after finishing step (ii) in removing non-phosphopeptides(s) which do(es) not bind to TiO₂, an elution solution is transferred into each sample well (i.e. the inner area of the circular area presented in FIG. 1b) to conduct the elution procedure of step (iii). Then, the plate is dried and a suitable MALDI-TOF matrix solution is added into each sample well to directly conduct a MALDI-TOF experiment to analyze the purified phosphopeptide(s) without loading the purified phosphopeptide sample onto another MALDI-TOF plate.

In the plate with TiO₂ on its surface according to the present invention, the substrate can be regenerated through a simple method after being used. For example, the PDMS layer can be directly stripped manually to regenerate the substrate for reuse.

Because the plate with TiO₂ on its surface according to present invention may have multiple aggregations of TiO₂ particles, the plate can be used to purify multiple phosphopeptide samples in a single batch. In addition, as described above, because phosphopeptides can be purified by the plate with TiO₂ on its surface according to the present invention without the complicated centrifugation steps, the percentage of sample loss can be reduced. Therefore, as compared to purifying phosphopeptides using a traditional TiO₂ Tip, the plate with TiO₂ on its surface according to the present invention has advantages including high throughput, easy operation, low sample loss, high experimental reproducibility, and its substrates can be regenerated easily.

The present invention will be further illustrated in details with specific examples as follows. However, the following examples are provided only for illustrating the present invention and the scope of the present invention is not limited thereby. The scope of the present invention is shown as followed in the Claims.

### EXAMPLES

### Example 1

### Preparation of a Plate with TiO₂ on its Surface

First, 10 parts by weight of a PDMS elastomer base (Sylgard® 184 reagent A, purchased from Dow Corning, Inc., USA) and 1 part by weight of a PDMS elastomer curing agent (Sylgard® 184 reagent B, purchased from Dow Corning Inc., USA) were mixed to form a PDMS mixture solution. The PDMS mixture solution was coated onto a surface of a MALDI-TOF plate (purchased from Bruker Daltonics Inc., USA), optionally smoothed by a glass slide or roller (purchased from Bio-Rad Inc., USA), and dried in an oven at 80° C. for 60 minutes. Then, 10 mg of TiO₂ particles with a particle size of 5 μm (purchased from GL Sciences Inc. Japan) and 10 mg of TiO₂ particles with a particle size smaller than 150 nm (purchased from Sigma-Aldrich Inc., USA) were suspended in a 100 μl aqueous solution of 60% acetonitrile respectively and shaken to mix. The aqueous suspension of TiO₂ particles was sucked up at an amount of about 2 μl to 5 μl repeatedly by a pipette tip and dropped onto the PDMS layer to form droplets (in the dropping step, each droplet was spaced for a interval), and then dried in an oven at 80° C. for 10 minutes to accomplish the preparation of the plate with TiO₂ on its surface.

The above plate with TiO₂ on its surface was scanned by an optical scanner. The scanned image is shown as FIG. 1b, wherein the TiO₂ particles were immobilized in a form of aggregations on the surface of the PDMS layer, showing a white circular area on the scanned image. The diameter of the individual circular area is about 2.5 mm (see FIG. 1a).

### Example 2

### Purification of Phosphopeptides from a Single Protein Sample

### (1) Protein Digestion

Ovalbumin (purchased from Sigma-Aldrich Inc., USA) was dissolved in 50 mM ammonium carbonate solution and heated at 90° C. for 20 minutes. Dithiothreitol (DTT) (10 mM) was added into the sample and heated at 56° C. for 20 minutes. Iodoacetamide (IAA) (55 mM) was added into the sample and placed at 25° C. in dark for 30 minutes. Then, trypsin was added to the protein solution at an enzyme-to-substrate ratio of 1:50 (w/w) at 37° C. for 12 hours to hydrolyze the protein. The hydrolyzed peptide solution was dried in a centrifugal concentrator.

### (2) Purification of Phosphopeptides

The above peptide sample was dissolved in a loading buffer (containing 80% ACN, 2% TFA, and 20 to 200 mg/ml of DHB). The peptide solution (2 μl) was sucked up by a pipette tip and dropped onto the aggregations of the TiO₂ particles of the plate which was prepared in Example 1, and incubated for a time period ranging from 2 minutes to 5 minutes. Then, the aggregations of the TiO₂ particles were washed with a wash solution (80% ACN, 2% TFA) to remove the unbinding non-phosphopeptides. The phosphopeptide(s) which bind(s) the aggregations of the TiO₂ particles were eluted with 3 μl to 5 μl of 0.05% NH₄OH. After the aggregations of the TiO₂ particles were dried, a MALDI-TOF matrix solution (2 mg/ml DHB in 25% ACN, 1% phosphoric acid) was added onto the aggregations of the TiO₂ particles, followed by a MALDI-TOF analysis.

### (3) MALDI-TOF Analysis

The samples were analyzed by a MALDI-TOF/TOF-MS (Ultraflex III TOF/TOF, purchased from Bruker Daltonics Inc., Germany). Peptide mass calibration for MALDI-TOF was performed with a peptide calibration standard kit (purchased from Bruker Daltonics Inc.). Spectra were acquired as the following experimental parameters: reflector mode; 25 kV accelerating voltage; 26.3 kV reflector voltage; and 20 ns pulsed ion extraction time.

### (4) Results

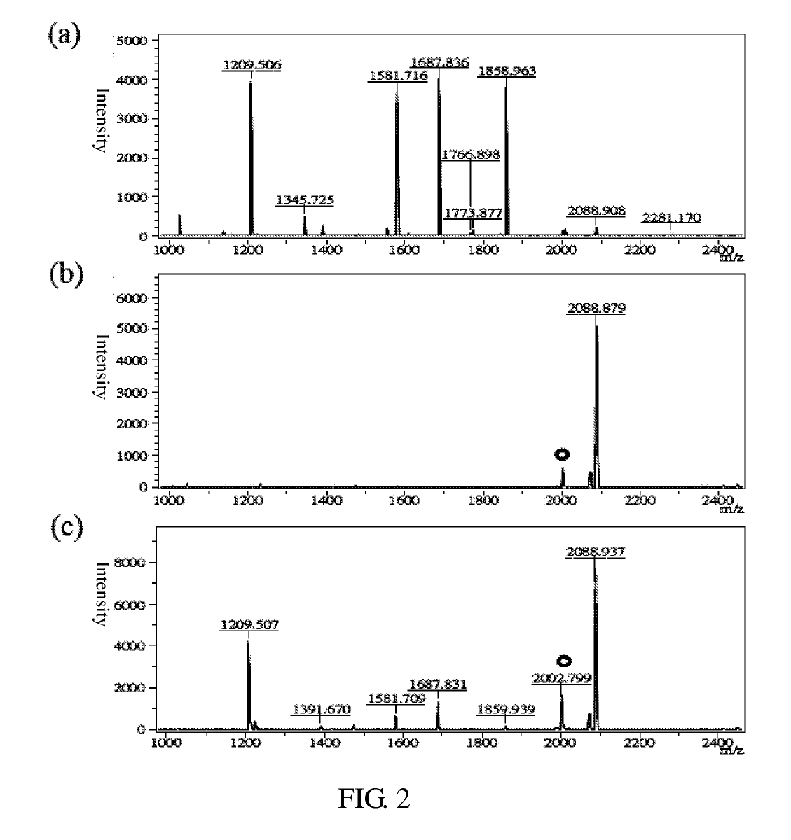

FIGS. 2a, 2b and 2c are mass spectra showing the MALDI-TOF analysis of the ovalbumin tryptic digests. FIG. 2a is a spectrum of unpurified ovalbumin tryptic peptides (control group), which shows many peak signals of unpurified ovalbumin peptides. The signal intensity of the peak signals of phosphopeptides (2088.908 m/z) was significantly lower than that of other major peak signals of non-phosphopeptides. FIG. 2b is a MALDI-TOF mass spectrum of ovalbumin digests which was purified by the plate with TiO₂ particles with a particle size of 5 μm on its surface, which shows a major peak signal of phosphopeptide (2088.879 m/z). FIG. 2c is a MALDI-TOF mass spectrum of ovalbumin which was purified by the plate with TiO₂ particles with a particle size smaller than 150 nm on its surface, which shows both the peak signal of phosphopeptide (2088.937 m/z) and other peak signals of non-phosphopeptides. The circle symbols (i.e., O) in FIGS. 2b and 2c represent the peak signal of a peptide that has lost a phosphopeptide fragment of 86 daltons.

As shown in FIGS. 2a to 2c, the plate with TiO₂ on its surface of the present invention can be used to effectively purify phosphopeptides. As compared to the plate using the TiO₂ particles in nano size, a plate with TiO₂ particles with a particle size of 5 μm on its surface can be used to purify phosphopeptides more effectively. On the other hand, when phosphopeptides were purified by a plate with TiO₂ with a particle size smaller than 150 nm on its surface, nonspecific binding will occur. It has been speculated that the nonspecific binding may be attributed to more abundant reacting sites for binding phosphopeptides and non-phosphopeptides in the nano-TiO₂ particles.

### Example 3

Three non-phosphorylated proteins (bovine serum albumin (BSA), myoglobin, and cytochrome C) and three phosphorylated proteins (ovalbumin, α-casein, and β-casein) (all purchased from Sigma-Aldrich Inc., USA) were dissolved into a 50 mM ammonium carbonate solution respectively to form a solution of protein mixture. Then, the proteins were digested by trypsin by using the method shown in Example 2. The tryptic peptides (20 fmole) of each six proteins were mixed, purified by the plate with TiO₂ on its surface (the particle size of TiO₂ particle is 5 μm) prepared in Example 1, and analyzed by the MALDI-TOF.

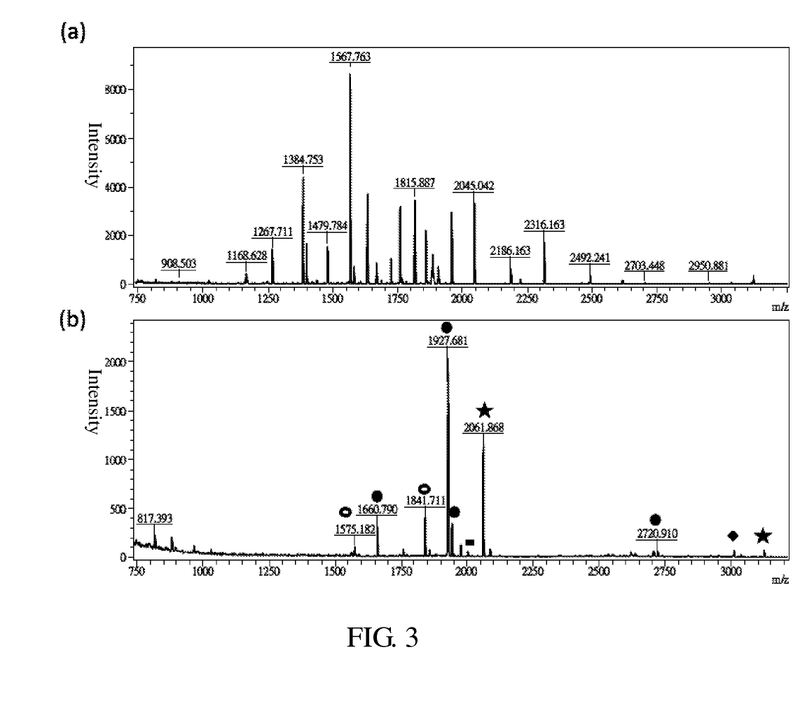

FIG. 3a is a mass spectrum showing the MALDI-TOF analysis of the solution of complicated peptides mixture without being purified by a plate with TiO₂ on its surface, wherein the peak signals of non-phosphopeptides were major peaks in the spectrum. FIG. 3b is a mass spectrum showing the MALDI-TOF analysis of the solution of complicated peptides mixture purified by the plate with TiO₂ on its surface, wherein the meanings of each symbol are represented below: “” represents a peak signal of a phosphopeptide of α-S1-casein; “♦” represents a peak signal of a phosphopeptide of α-S2-casein; “★” represents a peak signal of a phosphopeptide of β-casein; “▪” represents a peak signal of a phosphopeptide of ovalbumin; and “◯” represents the peak signal of a peptide that has lost a phosphopeptide fragment of 86 daltons.

Table 1 shows the results of the MALDI-TOF analysis of the solution of complicated peptides mixture which was purified or non-purified by the plate with TiO₂ on its surface of the present invention; the signal and noise ratio (S/N) of each phosphopeptide peak signal (four replicates were measured and averaged); and the phosphopeptides and phosphorylation sites (lowercase “p” in the sequence represents the phosphorylation site).

TABLE 1

| | | Peptide sequence | | | |
|---|---|---|---|---|---|
| | | (including the | | S/N before | S/N after |
| | | phosphorylation | SEQ ID | purifi- | purifi- |
| m/z | Phosphopeptide | sites) | NO. | cation | cation |
| 1927.69 | α-S1-casein (amino | DIG[pS]E[pS]TEDQ | 1 | n.d | 106.8 |
| | acids 58-73) | AMEDIK | | | |
| 1943.68 | α-S1-casein | DIG[pS]E[pS]TEDQ | 1 | n.d | 55.6 |
| | (58-73 amino | A[pM]EDIK | | | |
| | acids) | | | | |
| 1660.79 | α-S1-casein | VPQLEIVPN[pS]AE | 2 | 3.3 | 25.0 |
| | (121-134 amino | ER | | | |
| | acids) | | | | |
| 2720.91 | α-S1-casein | QMEAE[pS]I[pS][pS] | 3 | n.d | 23.2 |
| | (74-94 amino | [pS]EEIVPN[pS]VEA | | | |
| | acids) | QK | | | |
| 3008.01 | α-S2-casein | NANEEEYSIG[pS] | 4 | n.d | 18.1 |
| | (61-85 amino | [pS][pS]EE[pS] | | | |
| | acids) | | | | |
| 2061.83 | β-casein (48-63 | FQ[pS]EEQQQTEDE | 5 | n.d | 93.3 |
| | amino acids) | LQDK | | | |
| 3122.27 | β-casein (16-40 | RELEELNVPGEIVE[pS] | 6 | 36.8 | 84.85 |
| | amino acids) | L[pS][pS][pS]EES | | | |
| | | ITR | | | |
| 2088.89 | Ovalbumin | EVVG[pS]AEAGVD | 7 | n.d | 17.6 |
| | (340-359 amino | AASVSEEFR | | | |
| | acids) | | | | |

(n.d: not detected)

The results in FIG. 3a, FIG. 3b and Table 1 show that when the solution of peptides mixture was purified by the plate with TiO₂ on its surface of the present invention, its MS signals of phosphopeptide are largely enhanced, while the MS signals of non-phosphopeptide are nearly un-observable in the mass spectrum of FIG. 3b. The above results show that the plate with TiO₂ on its surface of the present invention actually can be used to efficiently purify phosphopeptides from a solution of multiple proteins mixture, and thus, can prevent the MS signals of phosphopeptides from being suppressed by non-phosphopeptides during MS analysis.

### Example 4

### Evaluation of the Recovery Rate of Samples

To evaluate the recovery rate of a phosphopeptide sample purified by the method of the present invention, two of the following 20 fmole phosphopeptides were mixed: (1) VNQIG(pT)LSESIK (SEQ ID NO:8), 1368.68 m/z; and (2) VNQIGTL(pS)E(pS)IK (SEQ ID NO:9), 1448.64 m/z (lowercase “p” in the sequence represents the phosphorylation site). The aforesaid phosphopeptides mixture was purified by the plate with TiO₂ on its surface (the TiO₂ particles with a particle size of 5 μm) of the present invention by the method shown in Example 2. The non-purified phosphopeptides mixture was used as the control group. Then, a purified peptide sample and an internal standard (2 fmole angiotensin II, 1046.54 m/z) were mixed and the MALDI-TOF analysis was conducted.

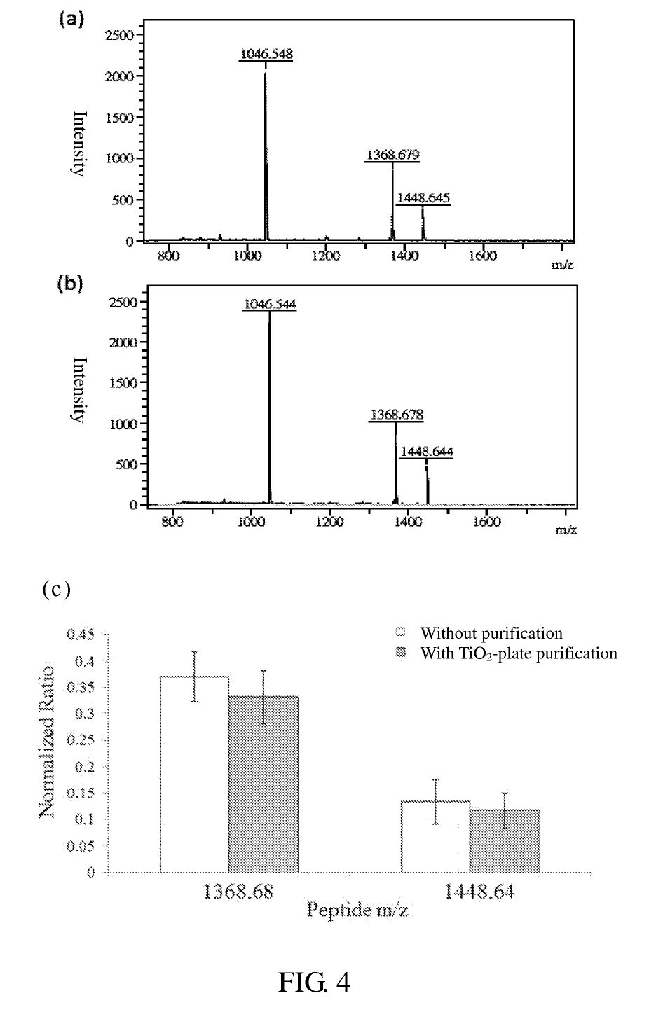

FIG. 4a is a mass spectrum showing the MALDI-TOF analysis of phosphopeptides mixture without purification (three replicates were measured and averaged). FIG. 4b is a mass spectrum showing the MALDI-TOF analysis of phosphopeptides mixture which was purified by the plate with TiO₂ on its surface (three replicates were measured and averaged). FIG. 4c is a bar diagram comparing the peak ratios of 1368.68/1046.54 m/z and 1448.64/1046.54 m/z of FIG. 4a and FIG. 4b, wherein the peak ratios of 1368.68/1046.54 m/z and 1448.64/1046.54 m/z of FIG. 4a are about 0.37 (STD, 0.05) and about 0.13 (STD, 0.04), respectively, and those of FIG. 4b are about 0.33 (STD, 0.05) and about 0.12 (STD, 0.03), respectively. Therefore, the recovery for the two 20 fmole phosphopeptides were about 90% and about 92%, respectively, illustrating a very low sample loss in dealing with such trace sample amounts.

### Example 5

### Evaluation of the Detection Limit of Samples

To evaluate the detection limit of the method of the present invention and compare said method to a traditional purification method, 2 fmole, 5 fmole, 10 fmole and 20 fmole β-casein were digested by trypsin respectively by the method shown in Example 2. Then, the samples were purified by the plate with TiO₂ on its surface (the TiO₂ particles with particle size of 5 μm), and analyzed by mass spectrometry.

The purification steps using the TiO₂ pipette tip were shown as follows: first, loading the TiO₂ beads suspended in 80% ACN and 0.1% TFA into a GELoader pipette, and producing air pressure by using a plastic injector to load the TiO₂ tip till 2 μm of height of TiO₂; loading the hydrolysed β-casein into the TiO₂ tip; washing with 25 μl of 80% ACN and 2% TFA solution and eluting phosphopeptides with 20 μl of 0.05% NH₄OH solution (pH 10.5); washing the column of TiO₂ tip with water; eluting the phosphopeptides with 10 μl of 50% ACN and 0.1 FA solution; drying the eluted solution by a centrifugal concentrator; dissolving the dried sample into 2 μl of MALDI-TOF matrix solution (2 mg/ml of DHB in 25% ACN and 1% phosphoric acid (PA)) and conducting MALDI-TOF analysis. The loading, washing, and elution steps of the aforesaid sample were conducted and operated by centrifugation (the method of manufacturing the TiO₂ tip can be seen in Larsen M R et al, Highly selective enrichment of phosphorylated peptides from peptide mixtures using titanium dioxide microcolumns. Molecular & cellular proteomics: MCP 2005; 4:873-86, which is entirely incorporated hereinto by reference).

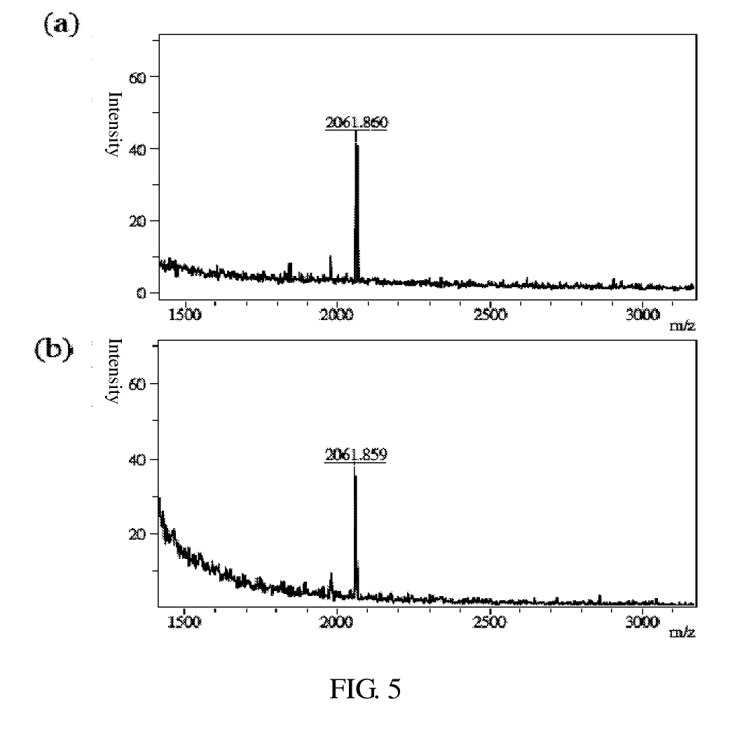

FIG. 5a is a mass spectrum showing the MALDI-TOF analysis of 2 fmole β-casein digested by trypsin and purified by the plate with TiO₂ on its surface according to the present invention, wherein the S/N of phosphopeptide peak of 2061.8 m/z is 14.7 (four replicates were measured and averaged), showing that the method of the present invention can be used to purify the samples in a very low concentration. FIG. 5b is a mass spectrum showing the MALDI-TOF analysis of 20 fmole β-casein hydrolysed by Trypsin and purified by a traditional TiO₂ Tip, wherein the S/N of phosphopeptide peak of 2061.8 m/z is 12.6 (three replicates were measured and averaged). The above results show that the method of purifying the phosphopeptides by the plate with TiO₂ on its surface of the present invention has a higher detecting sensitivity.

### Example 6

### Evaluation of the Sample Capacity

To evaluate the sample capacity for purified phosphopeptides of the plate with TiO₂ on its surface (the diameter of each TiO₂ circular area is about 2.5 mm) prepared in Example 1 of the present invention, β-casein samples (0.5 μg, 1 μg, 5 μg, 10 μg, 50 μg, and 100 μg) were purified by the plate with TiO₂ on its surface by the method described in Example 2 of the present invention, and then mixed with ACDH peptide samples (100 fmole, 200 fmole, 1 pM, 2 pM, 10 pM, and 20 pM) (adrenocorticotropic hormone fragment, having a sequence of RPVKVYPNGAEDESAEAFPLEF (SEQ ID NO:10), 2464.2 Da, act as an internal standard) respectively, and then analyzed by MALDI-TOF.

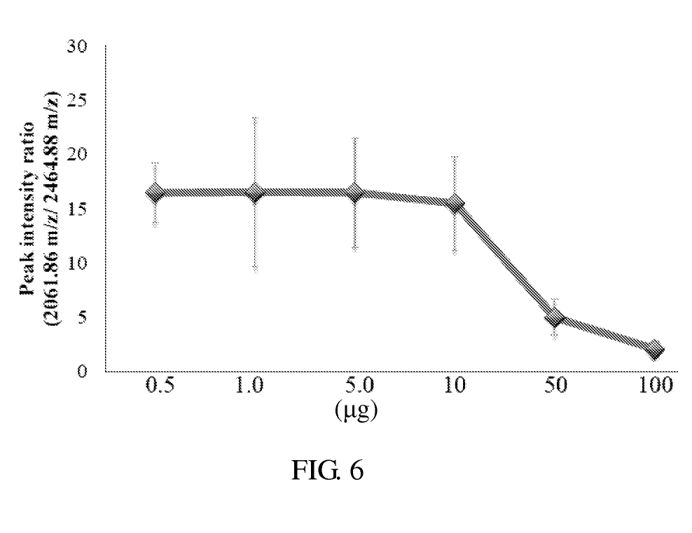

As shown in FIG. 6, when 0.5 μg to 100 μg β-casein were purified by the plate with TiO₂ on its surface of the present invention, the peak intensity ratios of 2061.8 m/z (the phosphorylated peak signal of (3-casein) to 2465.2 m/z (the internal standard) were all about 16, which means the protein amount that is applied is still below the capacity limitations of the plate with TiO₂ on its surface. When 10 μg of β-casein was purified by the plate with TiO₂ on its surface of the present invention, the peak intensity ratio of 2061.8 m/z to 2465.2 m/z slightly decreased to about 15. When 50 μg of β-casein was purified by the plate with TiO₂ on its surface of the present invention, the peak intensity ratio of 2061.8 m/z to 2465.2 m/z significantly dropped down to about 5. When 100 μg of β-casein was purified by the plate with TiO₂ on its surface of the present invention, the peak intensity ratio of 2061.8 m/z to 2465.2 m/z significantly dropped down to about 2. The above results show that when the diameter of each TiO₂ circular areas on the plate with TiO₂ on its surface of the present invention is about 2.5 mm, the sample capacity of each TiO₂ circular areas for β-casein purification is up to about 10 μg.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference, further description is deemed unnecessary. In addition, it should be understood that aspects of the invention and portions of various embodiments may be combined or interchanged either in whole or in part. Furthermore, those of ordinary skill in the art will appreciate that the foregoing description is by way of example only, and is not intended to limit the invention.

What is claimed is:

1. A plate with titanium dioxide on its surface, comprising:
(A) a substrate;
(B) a polydimethylsiloxane (PDMS) layer on at least one surface of the substrate; and
(C) one or more aggregations of titanium dioxide particles on the polydimethylsiloxane layer.

2. The plate as claimed in claim 1, wherein the titanium dioxide particles have a particle size ranging from 0.5 μm to 10 μm.

3. The plate as claimed in claim 1, wherein the titanium dioxide particles have a particle size ranging from 1 μm to 5 μm.

4. The plate as claimed in claim 1, wherein the substrate is selected from the group consisting of a metal substrate, a glass substrate, a polymethacrylate substrate, a polycarbonate substrate, a polyethylene terephthalate substrate, and a wood substrate.

5. A method of manufacturing the plate with titanium dioxide on its surface as claimed in claim 1, comprising the following steps:
(a) providing a substrate;
(b) forming a polydimethylsiloxane layer on at least one surface of the substrate;
(c) applying an aqueous suspension of titanium dioxide particles onto at least a portion of the polydimethylsiloxane layer; and
(d) drying the applied titanium dioxide suspension thereby forming one or more aggregations of titanium dioxide particles on the polydimethylsiloxane layer.

6. The method as claimed in claim 5, wherein the step (c) comprises dropping the aqueous suspension on the polydimethylsiloxane layer to form one or more individual droplets of the aqueous suspension.

7. The method as claimed in claim 5, wherein the titanium dioxide particles have a particle size ranging from 0.5 μm to 10 μm.

8. The method as claimed in claim 7, wherein the titanium dioxide particles have a particle size ranging from 1 μm to 5 μm.

9. The method as claimed in claim 5, wherein the aqueous suspension further comprises an organic solvent having a polarity lower than that of water.

10. The method as claimed in claim 9, wherein the organic solvent is selected from the group consisting of acetonitrile, methanol, ethanol, n-propanol, isopropanol, n-butanol, isobutanol, acrylonitrile, acetone, ethyl acetate, tetrahydrofuran, dichloromethane, trichloromethane, benzene, methyl benzene, n-hexane, n-pentane, and combinations thereof.

11. The method as claimed in claim 5, wherein the drying in the step (d) is conducted at a temperature ranging from 10° C. to 100° C.

12. The method as claimed in claim 11, wherein the drying in the step (d) is conducted at a temperature ranging from 20° C. to 80° C.

13. The method as claimed in claim 5, wherein the substrates is selected from the group consisting of a metal substrate, a glass substrate, a polymethacrylate substrate, a polycarbonate substrate, a polyethylene terephthalate substrate, and a wood substrates.

14. A method for purifying phosphopeptides, comprising the following steps:
(i) contacting a phosphopeptide-containing solution with the aggregations of titanium dioxide particles on the plate as claimed in claim 1 for a time period ranging from 1 minute to 60 minutes;
(ii) rinsing the aggregations of titanium dioxide particles with a wash solution; and
(iii) contacting the aggregations of titanium dioxide particles with an elution solution for a time period ranging from 1 minute to 30 minutes to elute phosphopeptide(s).

15. The method as claimed in claim 14, wherein the wash solution in the step (ii) comprises an organic phase and an acid.

16. The method as claimed in claim 15, wherein the organic phase is selected from the group consisting of acetonitrile, methanol, ethanol, n-propanol, isopropanol, n-butanol, isobutanol, acrylonitrile, acetone, ethyl acetate, tetrahydrofuran, dichloromethane, trichloromethane, benzene, methyl benzene, n-hexane, n-pentane, and combinations thereof; and the acid is selected from the group consisting of a substituted or unsubstituted formic acid, a substituted or unsubstituted acetic acid, and combinations thereof.

17. The method as claimed in claim 16, wherein the organic phase is acetonitrile and the acid is formic acid.

18. The method as claimed in claim 14, wherein the elution solution in the step (iii) is selected from the group consisting of an ammonia solution, an aqueous solution of an ammonium salt, an aqueous solution of formic acid, and combinations thereof.

19. The method as claimed in claim 18, wherein the elution solution in the step (iii) is an ammonia solution.

20. The method as claimed in claim 14, wherein the time for contacting in the step (i) is 2 minutes to 20 minutes, and the time for contacting in the step (iii) is 2 minutes to 10 minutes.


SEQUENCE LISTING (10 sequences)

SEQ ID NO: 1 - protein, 16 aa, Artificial sequence
  alpha-S1-casein, 58-73 a.a.
DIGSESTEDQAMEDIK

SEQ ID NO: 2 - protein, 14 aa, Artificial sequence
  alpha-S1-casein, 121-134 a.a.
VPQLEIVPNSAEER

SEQ ID NO: 3 - protein, 22 aa, Artificial sequence
  alpha-S1-casein, 74-94 a.a.
QMEAESISSSEEIVPNSVEAQK

SEQ ID NO: 4 - protein, 16 aa, Artificial sequence
  alpha-S2-casein, 61-85 a.a.
NANEEEYSIGSSSEES

SEQ ID NO: 5 - protein, 16 aa, Artificial sequence
  beta-casein, 48-63 a.a.
FQSEEQQQTEDELQDK

SEQ ID NO: 6 - protein, 25 aa, Artificial sequence
  beta-casein, 16-40 a.a.
RELEELNVPGEIVESLSSSEESITR

SEQ ID NO: 7 - protein, 20 aa, Artificial sequence
  Ovalbumin, 340-359 a.a.
EVVGSAEAGVDAASVSEEFR

SEQ ID NO: 8 - protein, 12 aa, Artificial sequence
  phosphorylated peptide
VNQIGTLSESIK

SEQ ID NO: 9 - protein, 12 aa, Artificial sequence
  phosphorylated peptide
VNQIGTLSESIK

SEQ ID NO: 10 - protein, 22 aa, Artificial sequence
  Adrenocorticotropic hormone fragment
RPVKVYPNGAEDESAEAFPLEF